(19) United States
(12) Patent Application Publication
(10) Pub. No.: US 2010/0129873 A1
(43) Pub. Date: May 27, 2010

(54) Highly Parallel Gel-Free Cloning Method

(76) Inventors: Chris Suh, San Jose, CA (US); Lee Hoang, San Jose, CA (US); Douglas T. Gjerde, Saratoga, CA (US); David P. Hornby, Widnes (GB)

Correspondence Address: PHYNEXUS, INC., 3670 CHARTER PARK DRIVE, SAN JOSE, CA, 95136, US

(21) Appl. No.: 12/625,535

(22) Filed: Nov. 24, 2009

Related U.S. Application Data
(60) Provisional application No. 61/117,537, filed on Nov. 24, 2008.

(51) Int. Cl. C12P 19/34

(52) U.S. Cl. 435/91.1

(57) ABSTRACT

A highly parallel method for gene cloning is presented. PCR products can be isolated using a solid phase and ligated into a positive selection vector. The cloning method has a very high success rate and can be performed entirely by a liquid handling robot with very little human intervention.

### CROSS REFERENCE TO RELATED APPLICATIONS

This application claims priority to and benefit of U.S. Provisional Patent Application No. 61/117,537 filed Nov. 24, 2008; U.S. patent application Ser. No. 10/620,155, now U.S. Pat. No. 7,488,603; and U.S. patent application Ser. No. 10/921,010, filed Aug. 17, 2004, the disclosures of which are incorporated herein by reference in their entirety for all purposes.

### FIELD OF THE INVENTION

This invention relates generally to molecular biology, genomics and proteomics and more particularly to highly parallel systems and methods for automated gene cloning.

### BACKGROUND OF THE INVENTION

High throughput cloning is useful for many applications. One application is the study of protein interactions within the cell. As of 2009, over 1000 microbial genomes have been sequenced and more are underway. These microbial genomes have been analyzed for their gene content however, to understand how cells work, thousands of protein-protein interactions must be studied. To examine protein-protein interactions, all the genes of a cell must be cloned into vectors for expression and purification. Researchers studying individual pathways or the complete cellular compliment of genes have a need to perform these experiments.

Structural genomics is another area that requires a high throughput cloning method. One large initiative undertaken by the biological community is the determination of high resolution structures for all proteins. These structural studies require the expression of large amounts of protein, which require an initial cloning step.

Another application for high-throughput cloning involves drug development in the biopharmaceutical industry. A potential protein or peptide drug molecule undergoes a development process including a maturation process in which the drug-target interaction is optimized for specificity and affinity. This process includes mutating key positions that will stabilize a structure or interaction. Alternatively positions causing steric hindrance are eliminated by mutating positions as well. PCR strategies can be used to introduce mutations at key positions.

While the data gathered from proteomics experiments are very useful, obtaining gene libraries from an organism remains a considerable challenge. A gene library is not a collection of random genome pieces. Instead, it requires cloning intact genes or operons (groups of genes). To be useful, the genes must be cloned in such a way that they can be expressed. If the genome sequence has been analyzed, individual genes or operons can be cloned by PCR. These cloning projects are extremely time-consuming and they are limited by the inability to sufficiently automate the cloning process. Many steps of the cloning process are still performed manually.

For example, after a gene of interest is amplified using PCR, purification of the PCR product for cloning is usually performed manually. The purified PCR product is then ligated into a cloning vector, a suitable host such as a bacterium is transformed and transformants are selected on an appropriate solid medium. The next step in the process requires growth and screening of individual colonies to find a clone carrying the gene of interest, which is also a manual and time-consuming process.

Despite advances in molecular biology, no automated, high-throughput, commercially available product exists for gene cloning. There is a need to automate the entire cloning process. But it is difficult to automate a process in which purification of PCR products is performed using gels and colony selection is performed on agar plates. The challenge to developing a high throughput cloning solution comes from finding alternatives to effective, yet inefficient methods.

Liquid handling robots can perform many of the processes required for cloning. When using these robotic systems, researchers must intervene at two steps during the process. First, the PCR products require purification in order to remove contaminants that will interfere with the cloning process. This process can involve casting an agarose gel, preparing the PCR product for loading into the gel, loading the gel, running the gel, staining the gel, visualization of the DNA bands by UV absorption, excision of the band of interest and extraction of the DNA from the gel.

The second step requiring manual intervention occurs when the user must identify the bacterial colony containing the clone of interest. Purified PCR products are often ligated into cloning vectors such as plasmids that carry genes conferring antibiotic resistance. Transformed bacterial cells are plated onto a selective medium containing an antibiotic and grown overnight. The colonies formed are then re-streaked onto fresh agar plates to generate single colonies that have arisen from a single cell. This step involves a second overnight incubation.

There are several strategies for identifying the correct clone. The single colonies can be picked and used to inoculate a liquid culture and grown to saturation, again overnight. Plasmid DNA can be prepared (miniprep) and the researcher can perform restriction digests or sequencing to confirm the clone carries the desired gene. Alternatively, positive clones can be identified by performing PCR directly on colonies. This method is faster than preparing the DNA but still requires plating and overnight growth.

In some cases bacterial cells carrying the cloned DNA are identified by their appearance or phenotype. For example X-gal, a modified sugar added to the culture medium, turns blue when hydrolyzed by beta-galactosidase. It is used as an indicator that cells have been transformed by plasmids containing an insert or DNA fragment. Since the insert disrupts the lacZ gene, bacterial colonies that have successfully acquired the insert DNA fragment will be white. Those bacterial colonies lacking the DNA insert will have a complete lacZ gene that produces beta-galactosidase and will turn blue in the presence of X-gal. In this example, recombinant host cells are selected on a medium containing an antibiotic. Next, they are screened for the presence of the correct insert DNA.

Another strategy is direct selection for clone identification or survival. In direct selection, only the correct recombinant can survive. The simplest example of direct selection occurs when the desired gene specifies resistance to an antibiotic (e.g. kanamycin). For example, the gene for kanamycin resistance can be cloned from plasmid R6-5 as follows. Plasmid R6-5 carries genes for resistance to several antibiotics. It is known that the kanamycin resistance gene lies within one of the 13 EcoRI fragments. To clone this gene, plasmid R6-5 is digested with the restriction enzyme, EcoRI and the resulting EcoRI fragments of R6-5 are inserted into the EcoRI site of a cloning vector such as pBR322. In this case, the kanamycin resistance gene can be used as the selectable marker. Transformants are plated onto a kanamycin-containing medium, on which the only cells able to survive and produce colonies are those recombinants that contain and express the cloned kanamycin resistance gene.

These existing methods are extremely tedious, even those methods that have introduced some automation into the process. Thus, there is a need for a completely automated cloning process. In the cloning methods described above, there are still non-liquid processes that require manual intervention, such as gel purification of PCR products and the growth of colonies. Any completely automated method must also automate these methods.

The automation of cloning carries the additional burden of error propagation. Any error in an early step of the process will be propagated and amplified as the process proceeds. Therefore, each step must have a very high success rate, close to 100%. Due to the problem of error propagation, it is not obvious that a multi-step, manual or partially automated cloning process can be fully automated, producing the desired clones at a very high success rate.

For example, if the first step has a success rate of 90% and the second step has a success rate of 95% and the third step has a success rate of 90% and the fourth step is 95% successful, then the product of the four steps is 0.90×0.95×0.90×0.95 which equals 0.73. That is, only 73% of the clones obtained will have the gene of interest.

In the manual cloning process, there are opportunities to correct errors during the process. For example, if the PCR does not produce a fragment of the expected length, a researcher can troubleshoot the PCR, and the reaction can be repeated. The process can in effect, reset the success rate for that step and error propagation is interrupted. So in a manual process, a low success rate for a given step can be corrected to bring the success rate back up to 100%. At that point, the next step of the process can proceed with little or no error propagation.

But a fully automated process must have a high success rate at each step, especially at the beginning of the process. Or, the process must have some way of eliminating the errors if they have occurred. There is no way to check the system so there can be no weak links in the process. Mathematically, the greatest danger is that errors occur early in the process because any error generated early will be multiplied at each successive step.

In a process such as cloning, there can be no guarantees that putting together different steps and completely automating the process can be successful. Even if each step of the process is described as being 100% successful, the result of an automated process cannot be certain of propagation of error. Automating a process which requires high level of success at each step is difficult and success cannot be predicted ahead of time.

Thus, there exists a need for a high throughput cloning method in which the hands-on intervention of researchers is eliminated, thus further automating the process. Additionally, there exists a need in which the process employs no gels or plating onto solid medium. Preferable, the entire process can be performed in a liquid state by a liquid handling robot. One final condition is placed on the automation process. There exists a need whereby the automation of steps does not propagate errors.

### BRIEF DESCRIPTION OF THE FIGURES

FIG. 1 depicts PCR 1 and PCR 2.

FIG. 2 depicts PCR 2 and capture of the PCR product on a pipette tip column.

FIG. 3 depicts release of the PCR from a pipette tip column into a well of a multi-well plate.

FIG. 4 depicts ligation and transformation of the purified PCR products.

FIG. 5 depicts the positive selection vector, pMTET1

### DESCRIPTION OF SPECIFIC EMBODIMENTS OF THE INVENTION

It is understood that the embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entireties for all purposes.

Unless defined otherwise, all technical and scientific terms used herein have the meaning commonly understood by a person skilled in the art to which this invention belongs. The following references provide one of skill with a general definition of many of the terms used in this invention: Dictionary of Microbiology & Molecular Biology, Paul Singleton and Diana Sainsbury, 3^{rd} edition, revised, ©2006, John Wiley and Sons; The Condensed Protocols from Molecular Cloning: A Laboratory Manual, Sambrook et al., ©2006, Cold Spring Harbor Laboratory Press, Cold Spring Harbor, New York; PCR Protocols (Methods in Molecular Biology), David Stirling and John M. S. Bartlett, ©2003, Humana Press, Inc.

The subject invention pertains to highly parallel gene cloning. In some embodiments, the genes to be cloned are amplified using PCR. A “gene of interest” is defined herein as a gene to be cloned using the subject invention. The term, “gene of interest” used interchangeably herein with the terms, “target DNA”, “target gene” and “sequence of interest”. After the PCR step, the gene is referred to herein as a “PCR product” or “DNA insert”. The polymerase chain reaction (PCR) is well known in the art of molecular biology and will not be described in detail here. PCR-based methods are a fast and convenient way of amplifying a large amount of a target gene of known sequence for cloning.

When PCR is used to amplify a target gene, the amplified gene must then be purified away from contaminants and reagents including genomic DNA, oligonucleotides primers, polymerase protein and PCR failure sequences. Traditionally, purification of PCR products is performed manually. One method involves agarose gel electrophoresis, staining the gel with ethidium bromide, visualization by UV, and excision of the band of interest using a razor blade or scalpel. To purify the PCR product from the gel, a kit, such as the Qiagen Gel Extraction Kit can be used. If the Qiagen Gel Extraction Kit is used, buffer is added to the excised agarose and it is heated to 50 degrees C. for 10 minutes until the agarose has melted. The sample is then applied to a spin column and a microfuge is used to spin the sample through. The spin column is washed with buffer and another centrifugation step is performed. The PCR product is then eluted with the addition of elution buffer followed by a spin.

Excision of the desired band from a gel has a couple of advantages. First, visualization of the amplified gene allows the researcher to confirm that the PCR product has the correct size. Second, this approach ensures that only the correct size PCR product is carried through to the cloning step. However, this method is tedious, time-consuming and impractical for cloning a large number of genes. Other techniques can be used for higher-throughput purification of PCR products. For example, filter plates can be used that allow the researcher to purify 96 PCR products simultaneously using centrifugation. This method will effectively purify the PCR product away from any remaining oligonucleotide primers and ddNTPs, but since the agarose gel step is not performed, the method does not provide confirmation that the PCR product is of the expected size. The disadvantage of these methods is that significant human intervention is required.

In the subject invention, the gel purification step is eliminated and the process of purifying the PCR product is completely automated. In the subject invention, the PCR product is comprised of a tag or label which is used to capture the PCR product using a solid phase that binds the tag. The term “label” is defined herein as any moiety that can be attached to a PCR product and subsequently used to capture the PCR product. The terms “tag” and “label” are used interchangeably herein, as are the terms, “tagged” and “labeled”. Capture of the PCR product can be performed with a liquid handling robot. In some embodiments, the tag is present on an oligonucleotide primer, and it is incorporated into the PCR product during PCR. In other embodiments, the PCR product is labeled following the PCR, e.g., enzymatically. In some embodiments, the tag is incorporated into both ends of the PCR product while in other embodiments, only one end is labeled.

In certain embodiments, restriction enzyme sites are also engineered into the PCR primers. Restriction sites in the resulting PCR products can then be cleaved to release the PCR product from the solid phase and also for subsequent cloning. When restriction sites are not incorporated into the PCR products, other ligation-independent cloning methods such as homologous recombination can be used to introduce the PCR products into a cloning vector.

A specific embodiment is now described in which PCR is used to incorporate a label into the amplified gene of interest. In this embodiment, restriction sites are additionally incorporated into both ends of the PCR product.

### PCR

The PCR strategy is designed to incorporate a label that can be used to purify the PCR product away from other species present in the PCR such as template DNA, dNTPs, polymerase, and PCR failure products. Any label can be incorporated that can be used to subsequently capture the PCR product. The PCR strategy is also designed to incorporate restriction sites into the PCR product. PCR primers for 96 genes of interest can be designed automatically (using software) and ordered in 96-well plates.

Any restriction site can be incorporated into a PCR primer. In some embodiments, restriction enzyme sites for EcoRI, HindIII or BamHI are incorporated into the PCR primers. A complete list of restriction enzyme sites can be found in the New England BioLabs catalog which is incorporated by reference herein or on the New England BioLabs website, www.neb.com.

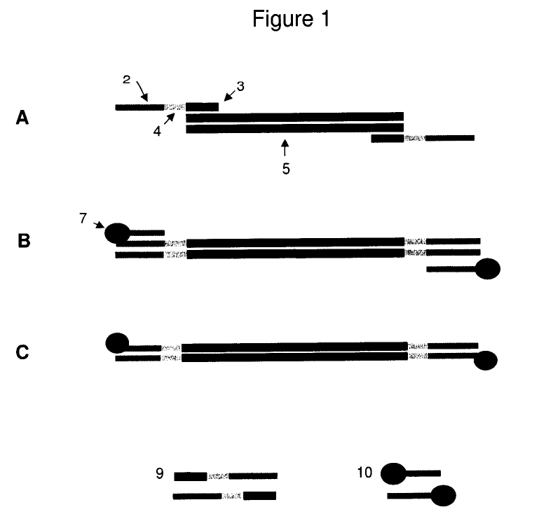

In some embodiments, a single PCR is used to amplify the gene of interest and incorporate a label. However, to attach a label to the PCR product in a cost effective manner, some embodiments of the invention utilize two rounds of PCR performed with two different sets of primers. These two rounds are referred to herein as PCR 1 and PCR 2 and are exemplified in FIG. 1. PCR 1 (FIG. 1A) generates copies of the target DNA with engineered sequences added to the 5′ and 3′ ends using unique primers 9 that contain sequences 3 complementary to the ends of target DNA 5. Primers 9 include universal sequence 2 and can also include DNA restriction sites 4. The universal sequence provides a template for the second round of PCR. The universal sequence is defined herein as any sequence complementary to the PCR 2 primers.

The PCR 1 primers must be carefully designed to ensure amplification of a single, unique target gene and to incorporate sequences needed for PCR 2 (FIG. 1B) and for restriction enzyme digestion. Failure to design primers unique to the target gene will result in the possibility of a heterogeneous mixture of clones. A person of ordinary skill knows how to design such primers. Though the majority of the PCR will likely consist of the desired product 8, a few side products can result. In FIG. 1, the PCR 1 primers contain the restriction site, but alternatively, the restriction site can be incorporated into the PCR 2 primers.

The restriction sites within the PCR primers must be carefully selected such that the corresponding restriction enzymes do not cleave the PCR product. In some embodiments, a site for a single restriction enzyme will be engineered into both ends of the PCR product. In other embodiments, two different restriction sites will be used, one at each end of the PCR product such that the subsequent cloning of the PCR product into a vector will be directional. Directional cloning refers to a process by which two different restriction enzymes create non-complementary sticky ends at either end of the PCR product. This allows the insert to be ligated into the vector in a specific orientation and also prevents self-ligation of the vector or the PCR product. In yet another embodiment, the primers can contain a multiple cloning site which is a sequence containing a series of restriction sites. A multiple cloning site allows the user to select from several restriction enzymes depending on the restriction sites present in the cloning vector and on the desired cloning strategy.

The template DNA used for PCR 1 can be from any source. It can be purified genomic or extra-chromosomal DNA. Whole cells or viruses can also be used as the PCR 1 template. Alternatively, the PCR 1 template can be heterogeneous, e.g. a mixture of cells or DNA. For example, the PCR 1 template can be a library such as a cDNA library so that the PCR products produced are from multiple individuals or clones.

In PCR 1, multiple copies of the target gene are generated using the PCR 1 primers mixed with a DNA polymerase, template DNA, and dTNPs. The product from PCR 1 serves as the template for PCR 2 (FIG. 1B). Following PCR 1, many copies of the target DNA are present, the ends of which contain the universal sequence. PCR 2 is performed using primers complimentary to the universal sequence. The PCR 2 primers 10 additionally contain label or tag 7 (such as biotin) which make the product of PCR 2 amenable to column purification (FIG. 1C).

In some embodiments, a low-fidelity PCR strategy can be used. For example, the same gene encoding a peptide or protein of interest can be provided in all 96 wells of a plate. Low fidelity PCR can be used to introduce mutations into different regions of the gene. After cloning, the proteins can be expressed and structure-function studies can be performed.

### Capture and Release Using a Solid Phase

Any tag or label can be used that allows for subsequent capture of the PCR product. In the subject invention, the label must bind the solid phase. A person skilled in the art can select tags and design strategies for labeling the gene of interest with the tag. A person skilled in the art can additionally design strategies to capture the labeled gene using an appropriate solid phase. For example, the label can be a biotin molecule and the solid phase can contain a streptavidin. In this strategy, PCR can be performed with oligonucleotide primers carrying a biotin label so that the biotin is incorporated into each end of the PCR product during PCR. A solid phase comprised of streptavidin will bind the biotinylated PCR product. Alternatively, the label can be a nucleic acid sequence captured by a solid phase comprised of the complementary sequence. The label can be a fluorescent label such as Cy3 or Cy5 captured by a solid phase comprised of an antibody specific to the fluorescent label. An amino acid tag, such as a 6× His tag can be captured with a metal chelate resin such as Ni-NTA. Other amino acid tags include GST, FLAG, and HA. A thiol tag can be used, forming a disulfide bond to a solid phase and subsequently releasing the captured PCR product with a reducing agent.

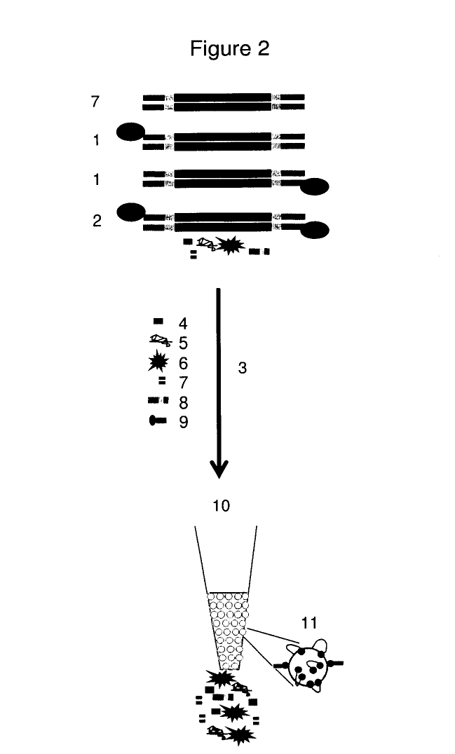

Similarly, any suitable solid phase in any format can be used to capture the PCR product. The term, “solid phase” is defined herein as any solid that can be used to capture a PCR product. In FIG. 2, the solid phase is a chromatography medium contained within a pipette tip column. The term “pipette tip column”, as used herein, refers to any column adapted to engage a liquid handling robot and is not restricted to the size or shape of commercially-available pipette tips. In this embodiment, a plurality of pipette tips can be attached to a liquid handling robot and the PCR can be aspirated into the pipette tip column through the lower end.

In other embodiments, the solid medium is not contained within a pipette tip column, but is provided in a different format amenable to use with a liquid handling robot. Examples of other formats include magnetic beads, loose chromatography beads, or media contained within a multi-well plate.

The solid phase can be any medium as long as it can be used in the capture step. Any type of binding or interaction between the label and solid phase can be exploited. Non-limiting examples include affinity, nucleic acid hybridization, covalent bond formation, normal phase, reverse phase and hydrophobic interaction.

FIG. 2 depicts capture of the labeled PCR product by solid phase bead 11 within pipette tip column 10. Efficient cloning requires that PCR 2 product (reference no. 2) is separated from minor PCR products 1 and PCR reagents such as dNTPs 4, template DNA 5, proofreading DNA polymerase 6, PCR 1 primers 8, and biotin-labeled PCR primers 9. Labeled PCR products 2 are retained by the solid phase, while unlabeled species are not. A close-up of solid phase bead 11 shows immobilization of both PCR 2 product (reference no. 2) and un-reacted biotin-labeled oligos 9. Partially-labeled species 1 can also bind the solid phase (not shown) but these should be very minor constituents.

After the labeled PCR product is captured, it can be desirable to rinse the solid phase to remove unbound species. The rinse can be any solution including buffer or water. Unlabeled, and hence un-retained species include product 7 of PCR 1, template DNA 5, DNA polymerase 6, dNTPs 4 and PCR 1 primers 8. If the solid phase is contained in a pipette tip, the rinse step can be performed by a liquid handling robot aspirating the rinse solution into the pipette tip column through the lower end.

A variety of strategies can be used to release the PCR product from the solid phase. A person skilled in the art can design such strategies. Release is often accomplished with an appropriate elution solution although a change in temperature (such as heating) can also be used. Elution solutions can use any mechanism to release the PCR product. In some embodiments, restriction enzyme-mediated release is used. In other embodiments, the PCR product is released from the solid phase by changing the pH.

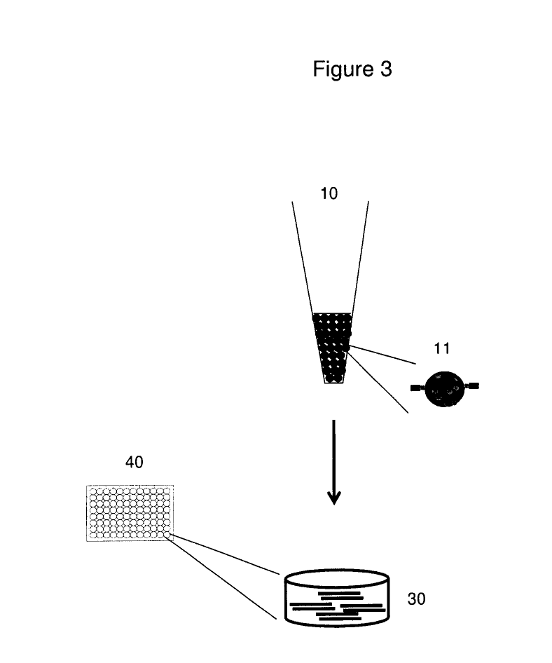

In FIG. 3, restriction enzyme-mediated release is used to release the PCR 2 product from bead 11 within column 10 into well 30 which resides in plate 40. The purified PCR 2 product is eluted from the column using restriction enzymes that cleave the specific sequences introduced during PCR 1. This on-column digest releases only the PCR product and leaves the biotin-labeled oligos bound to the column. Although, FIGS. 2 and 3 illustrate a high throughput purification procedure employing columns, other types of automated purification systems using solid phase extraction media can be used including magnetic beads and plates. When restriction enzyme-mediated release is used, it may be desirable to inactivate the restriction enzymes with a heating step prior to the subsequent ligation step.

### Ligation in a Positive Selection Cloning Vector and Transformation

The next step involves cloning the PCR product into a positive selection cloning vector. A “positive selection vector” is defined herein as a cloning vector in which successful cloning of a DNA results in an obvious phenotype. A positive selection vector can survive its host cells under the specific condition in which foreign DNA fragments are inserted into the vector. Thus, transformants harboring uncut or self-ligated vector cannot grow. In many positive selection vectors, a cytotoxic gene is interrupted by successful cloning of an insert so that transformed cells are viable only when an insert is successfully cloned into the vector.

In some embodiments the positive selection vector is linearized prior to ligation with a PCR product. When a cloning strategy involving restriction sites is used, the vector can be linearized with the same restriction enzymes that were used for enzyme-mediated release from the solid phase. In some embodiments, both ends of the purified PCR product have single-stranded overhangs (sticky ends). In other embodiments, one or both ends are blunt. In still other embodiments, the insert DNA is introduced into the vector by homologous recombination. In these embodiments, the vector can be linearized or circular.

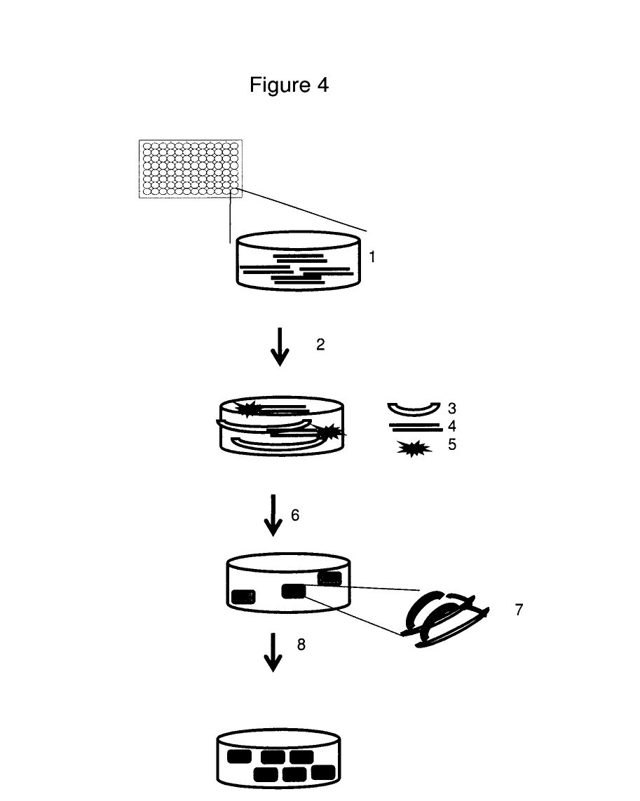

An embodiment detailing ligation of the purified PCR product into a self-propagating bacterial vector is shown in FIG. 4. Well 1 from a 96-well plate contains the purified PCR product. The PCR product was released from the solid phase using restriction enzyme-mediated release. In step 2, ligation of linearized vector 3 with the PCR product is catalyzed by DNA ligase 5. The PCR product cloned into the positive selection vector is referred to herein as the recombinant vector.

After ligation, the recombinants are transformed into a suitable host to create transformed host strains. In some embodiments a bacterial host such as E. coli or B. subtilis is used. Transformation methods are well known to those skilled in the art. As an example, E. coli cells can be subjected to heat-shock and incubated with the ligation mix at 37° C. for one hour. Any transformation method can be used as long as it can be performed in an automated fashion using a liquid handler. Step 6 of FIG. 4 depicts the transformation step. The dark cell in the center has acquired the recombinant vector. To select for cells that have acquired the vector, cells are grown in the presence of an antibiotic (step 8), which eliminates untransformed cells. The term, “transformed host strain” is defined herein as a collection of cells that harbor a recombinant plasmid or ligation products. The term, “transformed host cells” refers to individual within the transformed host strain.

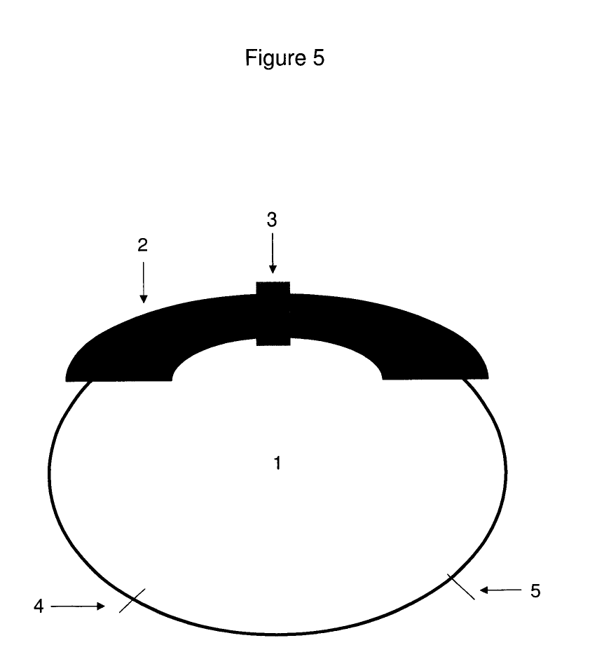

The positive selection vector has the advantage that only host cells harboring recombinant vectors can survive. Positive selection vector pMTET1 is shown in FIG. 5 (reference number 1). Multiple cloning site 3 on the vector is positioned to disrupt gene 2 that encodes a protein toxic to the cell. This cytotoxic gene product is the cytosine-specific DNA methyltransferase MspI. The M.MspI gene encodes an enzyme that methylates the outer cytosine on both strands of the sequence CCGG. Methylated cytosines are recognized and cleaved by the protein product of the mrcBC gene (reference number 4). The vector also carries gene 5 conferring tetracycline resistance. The product of this gene confers tetracycline resistance by excluding the antibiotic through a membrane pump. When the M.MspI gene is inactivated by insertion of the PCR product, recombinant cells can be isolated in a liquid medium containing tetracycline. If the vector doesn't ligate with the PCR product, the M.MspI gene is not inactivated. As a consequence, genomic DNA is methylated and then cleaved by the protein product of the mrcBC gene 4, resulting in cell death. Some host strains such as the E. coli strain DH5α have the mrcBC gene coded in the genome. For host strains that lack this gene, it can be provided on a cloning vector.

Because only cells harboring the recombinant vector survive this transformation, the need to plate out colonies is eliminated. The term, “selective conditions” is defined herein as those conditions under which only a transformed strain will grow. In the lacZ system described above, both blue and white colonies are selected on a medium containing antibiotic. The white colonies containing cloned inserts are then screened for the presence of the desired insert. A positive selection cloning vector further improves cloning efficiency and eliminates the screening step. When a positive selection cloning vector is used, only those cells containing the vector with a cloned insert will grow. This is particularly important for automated high-throughput cloning because the step of growing only white colonies is eliminated.

Any suitable positive selection vector can be used with the subject invention. Positive selection vectors are reviewed by Choi et al.¹, the contents of which are incorporated herein in their entirety. The positive selection vector can be modified to have a number of useful elements. For example, a T7 promoter allows for expression of the gene in a common bacterial expression system. The His tag gives the expressed protein a tag which can later be used for purification. ¹ Critical Reviews in Biotechnology, 22(3):225-244. 2002

### Verification

After the transformation step, it is desirable to verify that each well contains the desired clone. A person of skill in the art can perform verification using a number of techniques. In some embodiments, PCR is used for this verification step.

All the steps the cloning method can be performed in multi-well plates by a liquid handling robot. Multi-well plates can be any format including 96-well, 384-well or 1536-well plates. A liquid handling robot is defined herein as any machine that dispenses a selected quantity of reagent, samples or other liquid to a designated container.

Using the parallel gel-free cloning method with a positive selection vector allows elimination of a number of steps performed in traditional cloning. A comparison of this invention to traditional PCR cloning is listed in Table 1. Using the methods of the invention, the researcher can perform cloning in multi-well plates, either in parallel or in sequence using an automated liquid handler. A number of steps are eliminated and other steps are modified using the automated gel-free cloning method of the invention. A step-by-step comparison of the automated cloning procedure of the subject invention to traditional PCR cloning is shown in Table 1. The table shows one advantage of the automated invention over the classical traditional method is the complete elimination of three steps.

TABLE 1
Automated cloning compared to traditional cloning

| PCR cloning | | Gel-free cloning | |
|---|---|---|---|
| 1) | Design primers* | 1) | Design primers with “universal” site^{a} |
| 2) | PCR | 2) | PCR |
| 3) | Gel-electrophoresis* | 3) | Bioanalyzer to check PCR (optional)^{a} |
| 4) | Excise band* | | Step Eliminated |
| 5) | Extract target DNA* | 4) | Purify PCR product on column |
| 6) | Restriction digest | 5) | On-column restriction digest to release |
| 7) | Ligation | 6) | Ligation |
| 8) | Transformation | 7) | Transform with positive selection vector |
| 9) | Grow O/N | | Step Eliminated |
| 10) | Pick colonies* | | Step Eliminated |
| 11) | Grow O/N cultures | 8) | Grow O/N cultures |

^{a}Indicates steps requiring hands-on intervention
Gel-free method is done in liquid state allowing for complete automation

As shown in Table 1, the first steps, primer design and PCR, are performed differently in the two methods. Two rounds of PCR and two sets of primers are used for the automated cloning method. The first set of primers used for PCR 1 contains sequences complementary to the target DNA, sequences encoding a restriction enzyme site, and a universal sequence which is used to bind the PCR 2 primers. The PCR 2 primers contain a sequence complementary to the universal site and a label (such as biotin) used for purification of the PCR product. As an example, 96 genes can be cloned simultaneously in a 96-well plate using the automated cloning method. The PCR 1 primers will be unique to each gene and different PCR 1 primer sets will be used in each well. For PCR 2, the same PCR primers are used in each well. The PCR 2 primers hybridize to the universal sequence present on the PCR 1 primers. Because the automated cloning method requires two rounds of PCR, the total time for PCR is longer than the time required for a single PCR using the traditional cloning method.

In traditional cloning methods, PCR products are run on a gel, the proper size PCR product is selected, excised and purified from the gel. After the PCR products are purified, they can be digested with restriction enzymes for ligation into a vector.

But in the automated gel free cloning, PCR products are directly mixed with a solid phase for capture. If desired, yield of the PCR product can be checked using a bioanalyzer. Capture of the PCR product and the subsequent release (e.g., by restriction enzyme digest) are performed exclusively by the liquid handler. In some embodiments the column is a pipette tip column containing a solid phase that binds the PCR product. The restriction enzyme digest is then performed on the column. The pipette tip column can be attached to a liquid handler and the PCR reaction drawn into the column through the lower end. After the PCR product is bound to the column a wash step can be performed to eliminate unbound species such as template DNA, polymerase and dNTPs. Next a cocktail containing the restriction enzyme in the appropriate buffer can be drawn into the column. This eliminates the gel step used in the traditional method. This automated PCR clean up and restriction enzyme digest saves at least 4 hours when compared to the traditional method.

Following release of the digested PCR product from the column, the ligation and transformation steps are similar in the two methods. Ligation and transformation can also be performed in a multi-well format. Following the transformation, the traditional cloning method requires growing transformed cells on agar plates overnight followed by picking colonies and growing them again in liquid culture for overnight. By using a positive selection vector, the gel-free cloning method completely eliminates overnight growth on agar plates and the need to pick colonies. Instead, the transformed cells are directly grown overnight in liquid culture. Elimination of these steps saves at least 8 hours thus dramatically speeding up the entire cloning procedure. Furthermore, the automated gel-free cloning method requires less human intervention and therefore can be more easily scaled.

Tables 2 through 4 show a more detailed comparison illustrating time savings of using the automated cloning method over the traditional cloning method. The comparison is based on preparing and cloning 96 samples at a time. Table 2 lists the various steps along with a comparison of the time needed for each step. By using a positive selection vector, the gel free cloning method completely eliminates overnight growth on agar plates and the need to pick colonies. Instead, the transformed cells are directly grown overnight in liquid culture. Elimination of these steps saves more than 8 hours and speeds up the entire cloning procedure. Along with these advantages in time savings, the gel-free cloning method also requires less human intervention. In total, the gel free cloning method saves about 5 hours of hands-on time and saves 14 hours over the entire method.

Tables 3 and 4 show a simulation of how the methods would be applied to the work day. Note that since the automated method is faster, the entire method is repeated several times while two entire days are required for one pass of the manual method. The comparison is based on preparing and cloning 96 samples at a time. Increasing the number of samples with the gel method generally increases the amount of time needed by the same factor. This is because much of the time involved in the gel method is using manual techniques that can't be done in parallel. However, increasing the number of samples cloned using the gel-free technology will increase the time required very little, thus increasing productivity over the manual method even further. For example doubling the number of samples to 192 will increase the reagents used but will not significantly increase the total cloning time.

TABLE 2

| | | | Automated | | |
|---|---|---|---|---|---|
| Current Method | Time | | Method | Time | |
| 24 nuc Oligos | same | | 39 nuc Oligos $0.2/bp X2) | same | |
| ($0.2/bp X2) | | | | | |
| Biotin Labeled oligo | — | | Biotin labeled oligo | — | |
| PCR set up | 30 | m | PCR set up | 30 | m |
| PCR Rxn | 120 | m | PCR | 150 | m |
| Clean + Pour gel | 20 | m | | | |
| Polymerize gel | 30 | m | | | |
| Load sample (96) | 30 | m | | | |
| Run gel | 60 | m | | | |
| Excise band | 96 | m | | | |
| (1 min/sample) | | | | | |
| Melt gel + load sample + | 50 | m | Set up gel free cloning | 20 | m |
| vac | | | | | |
| Add PE + vac | 10 | m | Condition columns (auto) | 1 | m |
| Add EB + Wait + vac | 12 | m | Sample capture (auto) | 3 | m |
| Set up digest | 20 | m | Wash (auto) | 2 | m |
| Digest | 60 | m | On column digest Elute (auto) | 30 | m |
| Heat inactivation | 5 | m | Heat inactivation (auto) | 5 | m |
| Set up ligation | 20 | m | Set up ligation (auto) | 2 | m |
| Ligation | 10 | m | Ligation (auto) | 10 | m |
| Transformation/plating | 40 | m | Transformation (auto) | 40 | m |
| Incubate @ 37° C. (8 h or | 8 | h | | | |
| O/N) | | | | | |
| Pick 3-5 colonies/sample | 15 | m | | | |
| Grow @ 37° C. 8 h or O/N | 8 | h | Grow @ 37° C. 8 h or O/N | 8 | h |
| Mini prep (3 preps per | 90 | m | Mini prep (1 prep per sample | 30 | m |
| sample) | | | | | |
| Total (1678 m = 27.63 h) | 27.96 | h | Total (803 m = 13.386 h) | 13.38 | h |
| Total hands on time | 7.3 | h | Total hands on time | 2 | h |
| Total time savings | 14.58 | h | | | |
| Total hands on time | 5.3 | h | | | |
| savings | | | | | |

TABLE 3

| Current Method | Time | Automated Method | Time |
|---|---|---|---|
| Day 1 (start time) | 8:00 am | Day 1 (start time) | 8:00 am |
| Clean make gel during PCR | 8:50 am (50 m) | Set up + Run PCR 1 | 9:13 am (73 m) |
| | | (Includes digest + ligation) | |
| Sample loading | 9:20 am (30 m) | Transformation | 9:53 am (40 m) |
| Run gel | 10:20 am (60 m) | Second PCR (ready from | 10:00 am |
| | | O/N) | |
| Excise band (1 min/sample) | 11:56 am (96 m) | Set up + Run PCR 2 | 11:13 am (73 m) |
| | | (Includes digest + ligation) | |
| Qiagen gel extraction (for 96) | 1:08 pm (72 m) | Transformation | 11:53 am (40 m) |
| Set up digest + Digest + heat | 2:33 pm (85 m) | 3, 4, 5 PCR (ready from O/N) | 12:00 pm |
| inactivate | | | |
| Set up ligation + ligation | 3:03 pm (30 m) | Plates 3, 4, 5 done | 6:00 pm |
| Set + transformation + plating | 4:43 pm (40 m) | Grow O/N (all 5 plates) | |
| Day 2 (start time) | 8:00 am | Day 2 (starting time) | 8:00 am |
| Pick Colony + grow 8 h | 4:15 pm (8.25 h) | Mini prep (all 5 plates) | 10:30 am(150 m) |
| Mini prep | 5:45 pm (90 m) | | |

TABLE 4

| Current Method | Time | Automated Method | Time |
|---|---|---|---|
| Day 1 (start time) | 8:00 am | Day 1 (start time) | 8:00 am |
| PCR set up and PCR run | 10:30 am(150 m) | PCR set up and PCR run | 11:00 am(180 m) |
| | | (96X3) | |
| Clean make gel during PCR | 10:30 am (50 m) | Set up + Run PCR 1 | 12:13 pm (73 m) |
| | | (Includes digest + ligation) | |
| Sample loading | 11:00 am(30 m) | Transformation | 12:53 pm(40 m) |
| Run gel | 12:00 pm (60 m) | Second PCR (ready from | 1:00 pm |
| | | morning) | |
| Cut band (1 min/sample) | 1:36 pm (96 m) | Set up + Run PCR 2 | 2:13 pm (73 m) |
| | | (Includes digest + ligation) | |
| Qiagen gel extraction (for 96) | 2:48 pm (72 m) | Transformation | 2:53 pm(40 m) |
| Set up digest + Digest + heat | 3:58 pm (85 m) | Third PCR (ready from | 3:00 pm |
| inactiv | | morning) | |
| Set up ligation + ligation | 4:28 pm (30 m) | Set up + Run method | 4:13 pm (73 m) |
| | | (Includes digest + ligation) | |
| Set + transformation + plating | 5:08 pm (40 m) | Transformation | 4:53 pm(40 m) |
| Day 2 (start time) | 8:00 am | Grow O/N (all 3 plates) | |
| Pick Colony + grow 8 h | 4:15 pm (8.25 h) | Day 2 (starting time) | 8:00 am |
| Mini prep | 5:45 pm (90 m) | Mini prep (all three plates) | 9:30 am (90 m) |

### Examples

### Example 1

In this example, 96 E. coli genes are cloned. The entire method is performed by a liquid handling robot in a 96-well plate. PCR 1 primers specific for 96 full-length E. coli genes of interest are designed. One PCR 1 primer contains the sequence of an EcoRI restriction site and the other PCR 1 primer contains the sequence of a HindIII site. None of the 96 E. coli genes of interest contain an EcoRI or a HindIII site. Both PCR 1 primers contain a universal sequence that can be used as a template for PCR 2. Each PCR 2 primer contains a sequence that hybridizes to the universal sequence on the PCR 1 primers and each PCR 2 primer also contains a biotin label.

96 PCRs are performed using a thermal cycler integrated with a Tecan Freedom Evo liquid handling system. The template is E. coli genomic DNA. Following the PCR step, the 96 PCRs are aspirated into a 96 pipette tip columns, each column having a 5-μl bed. The medium within the columns contains streptavidin and the biotinylated PCR products bind the streptavidin columns. The columns are rinsed with a buffer to remove unbound species.

Next, a buffer containing HindIII and EcoRI is aspirated into the 96 columns and the digest is incubated on the pipette tip columns for 1 hour at 37° C., after which the solution is expelled from the columns, eluting the amplified genes. The amplified genes are then mixed with a positive selection vector that has been linearized with HindIII and EcoRI. The ligation reaction proceeds for 1 hour at 4° C.

After the ligation, the 96 ligation products are introduced into a suitable host. Competent E. coli cells are added to each well and the plate incubated at 37° C. The host cells will grow only if the ligation products contain a cloned insert.

To determine the cloning efficiency within a single well, transformed cells are plated onto a solid medium and grown overnight. DNA from 100 colonies is prepared and the insert is sequenced. All 100 colonies carry an insert. Ninety-eight of 100 clones carry the expected gene of interest. That is, 98% of the transformed cells within the well carry the gene of interest. The other 2 clones carry a shorter, unrelated DNA fragment.

To determine the cloning efficiency within the 96-well plate, transformed cells from each well are plated onto a solid medium and grown overnight. For each well, DNA is prepared from 2 colonies and the inserts are sequenced. Of the 96 wells, transformants from 95 wells carry the expected gene of interest. Calculating the success rate as a percentage, over 98% of the transformed host strains carry a gene of interest. One well contains a shorter insert, unrelated to the gene of interest. For each well, the 2 clones sequenced are identical.

### Example 2

96 E. coli genes are cloned as described in Example 1. The cloning efficiency within the 96-well plate is determined to be 100%. That is, when 2 clones from each well are sequenced, all 96 wells contain the expected gene of interest.

### Example 3

96 E. coli genes are cloned as described in Example 1, except magnetic beads comprised of streptavidin are used in place of a pipette tip column.

We claim:

1. A method for cloning a plurality of genes simultaneously comprising the steps of:
a. providing a plurality of PCR products, wherein each PCR product is comprised of at least one gene of interest and at least one label;
b. mixing the PCR products with a solid phase, wherein the label binds the solid phase;
c. releasing the PCR products from the solid phase;
d. ligating the PCR products into a positive selection vector to create a plurality of ligation products;
e. introducing each ligation product into a bacterial host strain to create a plurality of transformed host strains; and
f. growing the transformed host strains under selective conditions,
wherein steps (a) through (f) are performed by a liquid handling robot, and wherein at least 90 percent of transformed host cells carry a gene of interest.

2. The method of claim 1, wherein the method is further comprises the steps of amplifying a plurality of genes of interest using PCR primers to create a plurality of PCR products comprised of amplified genes of interest, wherein the PCR primers incorporate a label into the PCR products.

3. The method of claim 2, wherein the label is incorporated into each end of the PCR product.

4. The method of claim 2, wherein the label is biotin.

5. The method of claim 2, wherein each end of the PCR products is comprised of a restriction site.

6. The method of claim 2, wherein the solid phase is contained in a pipette tip column.

7. The method of claim 4, wherein the solid phase is comprised of streptavidin.

8. The method of claim 2, wherein the solid phase is contained in a multi-well plate.

9. The method of claim 1, wherein at least 95% of the transformed host cells carry a gene of interest.

10. A method for cloning a plurality of genes simultaneously comprising the steps of:
a. providing a plurality of PCR products, wherein each PCR product is comprised of at least one gene of interest and at least one label;
b. mixing the PCR products with a solid phase, wherein the label binds the solid phase;
c. releasing the PCR products from the solid phase;
d. ligating the PCR products into a positive selection vector to create a plurality of ligation products;
e. introducing each ligation product into a bacterial host strain to create a plurality of transformed host strains; and
f. growing the transformed host strains under selective conditions,
wherein steps (a) through (f) are performed by a liquid handling robot, and wherein at least 90 percent of transformed host strains carry a gene of interest.

11. The method of claim 10, wherein the method is further comprises the steps of amplifying a plurality of genes of interest using PCR primers to create a plurality of PCR products comprised of amplified genes of interest, wherein the PCR primers incorporate a label into the PCR products.

12. The method of claim 11, wherein the label is incorporated into each end of the PCR product.

13. The method of claim 11, wherein the label is biotin.

14. The method of claim 11, wherein each end of the PCR products is comprised of a restriction site.

15. The method of claim 11, wherein the solid phase is contained in a pipette tip column.

16. The method of claim 13, wherein the solid phase is comprised of streptavidin.

17. The method of claim 11, wherein the solid phase is contained in a multi-well plate.

18. The method of claim 10, wherein at least 95% of the transformed host cells carry a gene of interest.
